(19) United States
(12) Patent Application Publication
(10) Pub. No.: US 2019/0336194 A1
(43) Pub. Date: Nov. 7, 2019

(54) COMPENSATION ASSEMBLY FOR BALLOON CATHETER SYSTEM

(71) Applicant: Cryterion Medical, Inc., Carlsbad, CA (US)

(72) Inventors: Xiaoyu Yu, San Diego, CA (US); Ricardo Roman, San Diego, CA (US); Eugene J. Jung, San Diego, CA (US); Eric A. Schulheis, San Jose, CA (US); Chadi Hamouche, Saint-Laurent (CA)

(21) Appl. No.: 16/403,051

(22) Filed: May 3, 2019

Related U.S. Application Data
(60) Provisional application No. 62/666,964, filed on May 4, 2018.

(51) Int. Cl. A61B 18/02

(52) U.S. Cl. CPC A61B 18/02; A61B 2018/0022

(57) ABSTRACT

Various aspects of the present disclosure are directed toward apparatuses, systems, and methods that include a compensation assembly for a balloon catheter system. The balloon catheter system may include a balloon catheter having a guidewire lumen and a balloon that is secured to the guidewire lumen with the balloon being movable between a deflated state and an inflated state.

### CROSS-REFERENCE TO RELATED APPLICATION

This application claims priority to Provisional Application No. 62/666,964, filed May 4, 2018, which is herein incorporated by reference in its entirety.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter.

Various forms of energy can be used to ablate diseased heart tissue. These can include radio frequency (RF), cryogenics, ultrasound and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of the energy delivered is a critical factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation (AF) is one of the most common arrhythmias treated using catheter ablation. In the earliest stages of the disease, paroxysmal AF, the treatment strategy involves isolating the pulmonary veins from the left atrial chamber. Recently, the use of techniques known as “balloon cryotherapy” catheter procedures to treat AF has increased. In part, this stems from the balloon cryotherapy's ease of use, shorter procedure times and improved patient outcomes. Despite these advantages, there remains needed improvement to further improve patient outcomes and to better facilitate real-time physiological monitoring of tissue to optimally titrate energy to perform both reversible “ice mapping” and permanent tissue ablation.

In the case of balloon cryotherapy, one or more cryoballoons are maneuvered through the vascular system of the patient, and are ultimately positioned near or against targeted cardiac tissue. Once in position, the cryoballoons are inflated. Cryogenic fluid, such as liquid nitrous oxide, is delivered through a fluid injection line to an interior of the inflated cryoballoon(s) to cause tissue necrosis of the target cardiac tissue, which renders the tissue incapable of conducting electrical signals. Once the target tissue has been necrosed, the cryoballoons are then deflated and the balloon catheter is removed from the patient's body.

During repeated use of the cryoballoons in balloon cryotherapy, the cryoballoons go through multiple combinations of insertion, inflation, deflation and retraction. The cryoballoon is normally at its minimum profile prior to and during insertion. To be at its minimum profile, the cryoballoon needs to be deflated and fully extended. Inflation of the cryoballoon will typically shorten the distance between the distal end and the proximal end of the cryoballoon. This will cause the distal end of the cryoballoon to move proximally, the proximal end of the cryoballoon to move distally, or both.

Unfortunately, inflation of the cryoballoon(s) can cause undesired longitudinal movements or bowing/kinking for certain components, such as a guidewire lumen, if such movements are restricted within the medical device. Conversely, deflation of the cryoballoon(s) may in some situations only collapse the cryoballoon(s), and may not cause a longitudinal movement to return to its fully extended state. As such, deflation alone may not return the cryoballoon(s) to their minimum profile. Thus, it is also desired to assist the medical device to reduce the balloon profile as much as possible prior to repositioning or retraction of the medical device from the patient.

### SUMMARY

The present disclosure is directed toward a compensation assembly for a balloon catheter system, the balloon catheter system including a balloon catheter having a guidewire lumen and a balloon that is secured to the guidewire lumen, the balloon being movable between a deflated state and an inflated state. In various embodiments, the compensation assembly includes a housing, a slide and a fluid source. The housing has a housing interior. The slide is secured to the guidewire lumen. Additionally, the slide can be positioned within the housing interior and is selectively movable relative to the housing. The fluid source is in fluid communication with the balloon via a fluid conduit. The fluid source is controlled to selectively adjust a balloon pressure within the balloon so that the balloon moves between the deflated state and the inflated state. The balloon pressure moves the slide relative to the housing in a first direction when the balloon is moved from the inflated state to the deflated state. Additionally, the balloon pressure moves the slide relative to the housing in a second direction when the balloon is moved from the deflated state to the inflated state. In some embodiments, the first direction is substantially opposite to the second direction. Additionally, in certain embodiments, the housing interior includes a first interior region that is in fluid communication with the fluid source via the fluid conduit, and a second interior region that is at a reference pressure. In some such embodiments, the slide is positioned between the first interior region and the second interior region. In some embodiments, the fluid source selectively provides a negative balloon pressure that is lower than the reference pressure so that the balloon is moved toward the deflated state, thereby moving the slide relative to the housing in the first direction. Further, in such embodiments, the fluid source selectively provides a positive balloon pressure that is greater than the reference pressure so that the balloon is moved toward the inflated state, thereby moving the slide relative to the housing in the second direction.

Additionally, in certain embodiments, the housing includes a first stop that limits movement of the slide relative to the housing in the first direction, and a second stop that limits movement of the slide relative to the housing in the second direction.

The compensation assembly can further include a sealing element that seals a connection between the slide and the housing within the housing interior. In one embodiment, the sealing element is a pneumatic sealing element.

In some embodiments, at least a portion of the fluid conduit is positioned adjacent to the guidewire lumen.

In certain embodiments, the compensation assembly further includes a pressure sensor that is configured to sense the pressure within the balloon. The pressure sensor generates a pressure signal that is based on the sensed pressure. In some such embodiments, the compensation assembly further includes an actuator that is coupled to the guidewire lumen. Additionally, the compensation assembly can further include a controller that is electrically coupled to the pressure sensor and the actuator. The controller is configured to receive the pressure signal and to move the guidewire lumen based at least partially upon the pressure signal.

Additionally, in some embodiments, the present disclosure is directed toward a compensation assembly for a balloon catheter system, the balloon catheter system including a balloon catheter having a guidewire lumen and a balloon that is secured to the guidewire lumen, the balloon being movable between a deflated state and an inflated state, the compensation assembly including an actuator that is coupled to the guidewire lumen and moves the guidewire lumen between a first position and a second position; and a controller that controls the actuator such that the actuator moves the guidewire lumen between the first position and the second position so as to substantially coincide with the movement of the balloon between the deflated state and the inflated state.

The present disclosure is further directed toward a balloon catheter system including a balloon catheter having a guidewire lumen and a balloon that is secured to the guidewire lumen, and any embodiment of the compensation assembly as described above that selectively controls movement of the guidewire lumen relative to the housing. The balloon catheter system can further include a handle assembly that is configured to be used by an operator to control the balloon catheter. In one embodiment, the housing is positioned substantially within the handle assembly.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view illustration of a patient and one embodiment of a cryogenic balloon catheter system having features of the present disclosure, including a balloon catheter;

FIG. 2 is a simplified schematic side view illustration of a portion of the patient and a portion of one embodiment of the cryogenic balloon catheter system including the balloon catheter and an embodiment of a compensation assembly;

FIG. 3A is a simplified schematic side view illustration of a portion of one embodiment of the balloon catheter illustrated in a deflated state including an embodiment of the compensation assembly;

FIG. 3B is a simplified schematic side view illustration of the portion of the balloon catheter illustrated in an inflated state including the compensation assembly illustrated in FIG. 3A;

FIG. 4 is a simplified schematic side view illustration of a portion of another embodiment of the balloon catheter illustrated in the inflated state including another embodiment of the compensation assembly;

FIG. 5A is a simplified schematic side view illustration of a portion of still another embodiment of the balloon catheter illustrated in the deflated state including still another embodiment of the compensation assembly;

FIG. 5B is a simplified schematic side view illustration of the portion of the balloon catheter illustrated in the inflated state including the compensation assembly illustrated in FIG. 5A;

FIG. 6A is a simplified schematic side view illustration of a portion of yet another embodiment of the balloon catheter illustrated in the deflated state including yet another embodiment of the compensation assembly;

FIG. 6B is a simplified schematic side view illustration of the portion of the balloon catheter illustrated in the inflated state including the compensation assembly illustrated in FIG. 6A; and

FIG. 6C is a simplified schematic side view illustration of the portion of the balloon catheter again illustrated in the deflated state including the compensation assembly illustrated in FIG. 6A.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a compensation assembly for a balloon catheter system, e.g., a cryogenic balloon catheter system. More specifically, this disclosure provides a compensation assembly to automatically control position and/or accommodate movements, e.g., longitudinal movements, of certain components of the balloon catheter system during inflation and deflation of the balloon catheter and to reduce the balloon profile prior to retraction. In some embodiments, the compensation assembly is provided in the form of a pressure-controlled and/or pressure-based compensation assembly. However, in other embodiments, the compensation assembly need not be restricted to a pressure-controlled and/or pressure-based compensation assembly.

Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation—specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound and laser energy, as non-exclusive examples. The present disclosure is intended to be effective with any or all of these and other forms of energy.

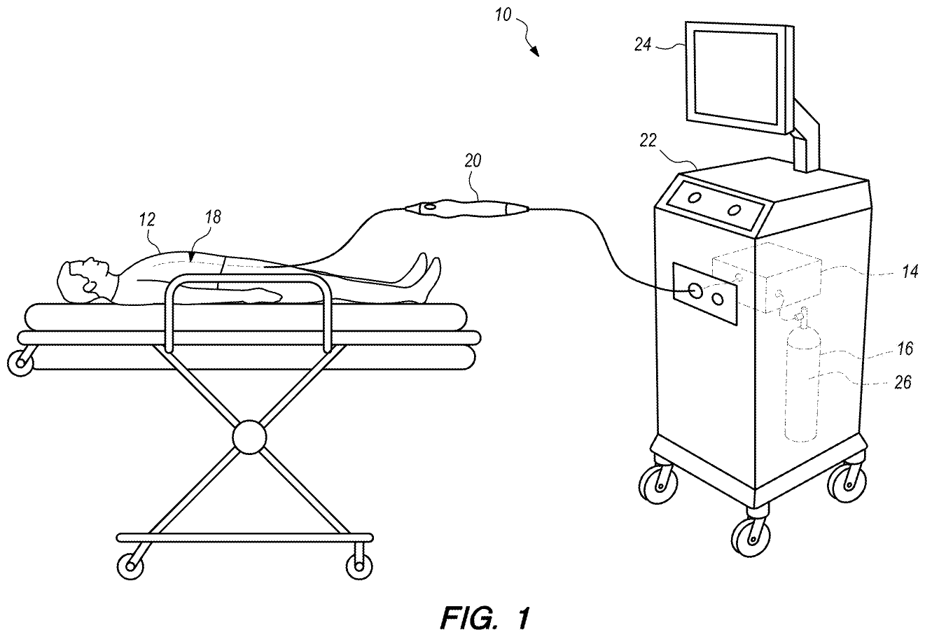

FIG. 1 is a simplified schematic side view illustration of an embodiment of a medical device 10 for use with a patient 12, which can be a human being or an animal. Although the specific medical device 10 illustrated and described herein pertains to and refers to a cryogenic balloon catheter system 10, it is understood and appreciated that other types of medical devices 10 or systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of a cryogenic balloon catheter system is not intended to be limiting in any manner.

The design of the cryogenic balloon catheter system 10 can be varied. In certain embodiments, such as the embodiment illustrated in FIG. 1, the cryogenic balloon catheter system 10 can include one or more of a control system 14 (illustrated in phantom), a fluid source 16 (illustrated in phantom), a balloon catheter 18, a handle assembly 20, a control console 22, and a graphical display 24.

It is understood that although FIG. 1 illustrates the structures of the cryogenic balloon catheter system 10 in a particular position, sequence and/or order, these structures can be located in any suitably different position, sequence and/or order than that illustrated in FIG. 1. It is also understood that the cryogenic balloon catheter system 10 can include fewer or additional components than those specifically illustrated and described herein.

In various embodiments, the control system 14 is configured to monitor and control various processes of the ablation procedure. More specifically, the control system 14 can monitor and control release and/or retrieval of a cooling fluid 26 (e.g., a cryogenic fluid) to and/or from the balloon catheter 18. The control system 14 can also control various structures that are responsible for maintaining and/or adjusting a flow rate and/or pressure of the cryogenic fluid 26 that is released to the balloon catheter 18 during the cryoablation procedure. In such embodiments, the cryogenic balloon catheter system 10 delivers ablative energy in the form of cryogenic fluid 26 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive data and/or other information (hereinafter sometimes referred to as “sensor output”) from various structures within the cryogenic balloon catheter system 10. In some embodiments, the control system 14 can receive, monitor, assimilate and/or integrate the sensor output, and/or any other data or information received from any structure within the cryogenic balloon catheter system 10 in order to control the operation of the balloon catheter 18. As provided herein, in various embodiments, the control system 14 can initiate and/or terminate the flow of cryogenic fluid 26 to the balloon catheter 18 based on the sensor output. Still further, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during a cryoablation procedure. Once the ablation procedure has initiated, the cryogenic fluid 26 can be delivered to the balloon catheter 18 and the resulting gas, after a phase change, can be retrieved from the balloon catheter 18, and can either be vented or otherwise discarded as exhaust. Additionally, the type of cryogenic fluid 26 that is used during the cryoablation procedure can vary. In one non-exclusive embodiment, the cryogenic fluid 26 can include liquid nitrous oxide. However, any other suitable cryogenic fluid 26 can be used. For example, in one non-exclusive alternative embodiment, the cryogenic fluid 26 can include liquid nitrogen.

The design of the balloon catheter 18 can be varied to suit the specific design requirements of the cryogenic balloon catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the control system 14. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue (not shown). While specific reference is made herein to the balloon catheter 18, as noted above, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16, and the graphical display 24. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In various embodiments, the handle assembly 20 can be used by the operator to initiate and/or terminate the cryoablation process, e.g., to start the flow of the cryogenic fluid 26 to the balloon catheter 18 in order to ablate certain targeted heart tissue of the patient 12. In certain embodiments, the control system 14 can override use of the handle assembly 20 by the operator. Stated in another manner, in some embodiments, based at least in part on the sensor output, the control system 14 can terminate the cryoablation process without the operator using the handle assembly 20 to do so.

The control console 22 is coupled to the balloon catheter 18 and the handle assembly 20. Additionally, in the embodiment illustrated in FIG. 1, the control console 22 includes at least a portion of the control system 14, the fluid source 16, and the graphical display 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the graphical display 24.

In various embodiments, the graphical display 24 is electrically connected to the control system 14. Additionally, the graphical display 24 provides the operator of the cryogenic balloon catheter system 10 with information that can be used before, during and after the cryoablation procedure. For example, the graphical display 24 can provide the operator with information based on the sensor output, and any other relevant information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

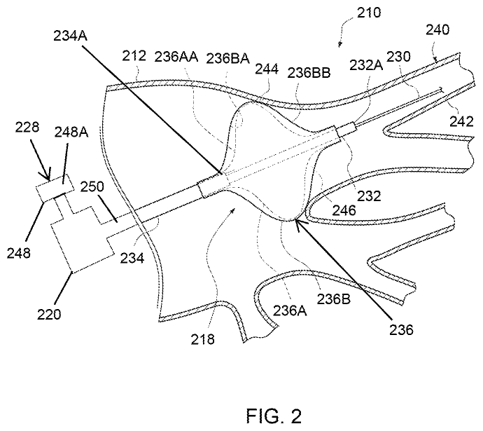

FIG. 2 is a simplified schematic side view illustration of a portion of the patient 212 and a portion of one embodiment of the cryogenic balloon catheter system 210. In this embodiment, the cryogenic balloon catheter system 210 can include one or more of the balloon catheter 218, the handle assembly 220 and a compensation assembly 228.

The balloon catheter 218 is inserted into the body of the patient 212 during the cryoablation procedure. The design of the balloon catheter 218 can be varied to suit the specific design requirements of the cryogenic balloon catheter system 210. In the embodiment illustrated in FIG. 2, the balloon catheter 218 includes one or more of a guidewire 230, a guidewire lumen 232, a catheter shaft 234, and one or more inflatable balloons 236 (hereinafter sometimes referred to as “balloons” or sometimes referred to generally as expandable elements). In one embodiment, the balloons can be cryoballoons. As shown in the embodiment illustrated in FIG. 2, the balloon catheter 218 can include two balloons, e.g., an inner balloon 236A (sometimes referred to herein as a “first balloon”) and an outer balloon 236B (sometimes referred to herein as a “second balloon”). As used herein, it is recognized that either balloon 236A, 236B can be described as the first balloon or the second balloon. Alternatively, the balloon catheter 218 can be configured to include only a single balloon 236. Additionally, it is understood that the balloon catheter 218 can include additional components or fewer components than those specifically illustrated and described herein. However, for the sake of clarity, these other structures have been omitted from the Figures.

As shown in the embodiment illustrated in FIG. 2, the balloon catheter 218 is configured to be positioned within the circulatory system 240 of the patient 212. The guidewire 230 and guidewire lumen 232 are inserted into a pulmonary vein 242 of the patient 212, and the catheter shaft 234 and the balloons 236A, 236B are moved along the guidewire 230 and/or the guidewire lumen 232 to near an ostium 244 of the pulmonary vein 242. In general, it is the object of the balloon catheter 218 to seal the pulmonary vein 242 so that blood flow is occluded. Only when occlusion is achieved does the cryothermic energy, e.g., of the cryogenic fluid 26 (illustrated in FIG. 1), cause tissue necrosis which, in turn, provides for electrically blocking aberrant electrical signals that trigger atrial fibrillation.

As shown, the guidewire lumen 232 encircles at least a portion of the guidewire 230. Additionally, the guidewire lumen 232 can be positioned or disposed at least partially within the catheter shaft 234, and can be movable within the catheter shaft 234. During use, the guidewire 230 is inserted into the guidewire lumen 232 and can course through the guidewire lumen 232 and extend out of a distal end 232A of the guidewire lumen 232. In various embodiments, the guidewire 230 can also include a mapping catheter (not shown) that maps electrocardiograms in the heart, and/or can provide information needed to position at least portions of the balloon catheter 218 within the patient 212.

As illustrated in this embodiment, the inner balloon 236A is positioned substantially, if not completely, within the outer balloon 236B. The specific design of and materials used for each of the one or more balloons 236 can be varied. For example, in some non-exclusive embodiments, the one or more balloons 236 can be formed from one or more of various grades of polyether block amides (PEBA), polyurethane, polyethylene terephthalate (PET), nylon, and other co-polymers of these materials. Alternatively, the one or more balloons 236 can be formed from other suitable materials.

Additionally, in some embodiments, the inner balloon 236A is bonded to a distal end 234A of the catheter shaft 234 and near the distal end 232A of the guidewire lumen 232. Further, one end of the outer balloon 236B may be bonded to a neck of the inner balloon 236A or to the distal end 234A of the catheter shaft 234, and the other end of the outer balloon 236B may be bonded to the guidewire lumen 232. It is appreciated that a variety of bonding techniques can be used and include heat bonding and adhesive bonding. Additionally, it is further appreciated that in embodiments that include only a single balloon 236, the balloon 236 can be secured to the catheter shaft 234, e.g., to the distal end 234A of the catheter shaft 234, and the guidewire lumen 232, e.g., near the distal end 232A of the guidewire lumen 232, in a similar manner. Alternatively, the one or more balloons 236 can be secured to other suitable structures.

During use, the inner balloon 236A can be partially or fully inflated so that at least a portion of the inner balloon 236A expands against at least a portion of the outer balloon 236B. Stated in another manner, during use of the balloon catheter 218, at least a portion of an outer surface 236AA of the inner balloon 236A expands and is positioned substantially directly against a portion of an inner surface 236BA of the outer balloon 236B. At certain times during usage of the cryogenic balloon catheter system 210, the inner balloon 236A and the outer balloon 236B define an inter-balloon space 246, or gap, between the balloons 236A, 236B. The inter-balloon space 246 is illustrated between the inner balloon 236A and the outer balloon 236B in FIG. 2 for clarity, although it is understood that at certain times during usage of the cryogenic balloon catheter system 210, the inter-balloon space 246 has very little or no volume. As provided herein, once the inner balloon 236A is sufficiently inflated, an outer surface 236BB of the outer balloon 236B can then be positioned within the circulatory system 240 of the patient 212 to abut and/or substantially form a seal with the ostium 244 of the pulmonary vein 242 to be treated.

As above, the handle assembly 220 is handled and used by the operator to operate, position and control the balloon catheter 218. Additionally, in the embodiment illustrated in FIG. 2, the handle assembly 220 can include at least portions of one or more of the guidewire 230, the guidewire lumen 232, and the compensation assembly 228, as described in greater detail herein.

As an overview, and as provided in greater detail herein below, in certain embodiments, the compensation assembly 228 includes a fluid source 248 that selectively delivers a pressurized fluid 248A, e.g., the cryogenic fluid 26 (illustrated in FIG. 1) or another suitable pressurized fluid, to the interior of the one or more balloons 236. Further, in some embodiments, the compensation assembly 228 selectively moves the pressurized fluid 248A to and/or from the fluid source 248 through a fluid conduit 250 to automatically and simultaneously compensate for necessary changes in length and/or positioning of the guidewire lumen 232 during operation of the cryogenic balloon catheter system 210, i.e. during selective inflation and deflation of the one or more balloons 236. Stated in another manner, in such embodiments, the compensation assembly 228 synchronizes the movement of the guidewire lumen 232 with the state of the one or more balloons 236, i.e. based on the positive (inflated) pressure or negative (deflated) pressure within the one or more balloons 236. Stated in still another manner, the compensation assembly 228 is configured to control the longitudinal position of the guidewire lumen 232 during inflation and deflation of the one or more balloons 236 by controlling a fluid pressure within the fluid conduit 250. As such, the cryogenic balloon catheter system 210 can be said to include an automated, pressure-controlled and/or pressure-based compensation assembly. Additionally, or in the alternative, in other embodiments, the compensation assembly 228 can be configured to compensate for necessary changes in length and/or positioning of the guidewire lumen 232 prior to and/or subsequent to any selective inflation and deflation of the one or more balloons 236. Further, or in the alternative, in still other embodiments, the compensation assembly 228 can utilize an actuator that is coupled to the guidewire lumen 232 to compensate for necessary changes in length and/or positioning of the guidewire lumen 232 with or without the use of pressurized fluid 248A from the fluid source 248.

In certain embodiments, at least a portion of the compensation assembly 228 is included and/or incorporated within the handle assembly 220. Alternatively, in other embodiments, the compensation assembly 228 can be provided separately from the handle assembly 220.

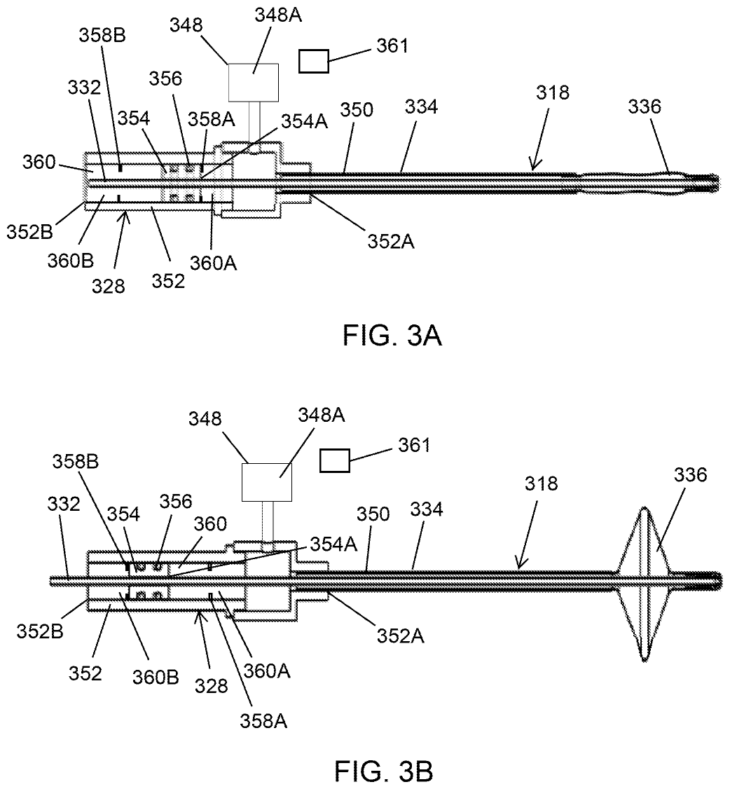

FIGS. 3A and 3B illustrate the design and operation of one non-exclusive embodiment of the compensation assembly 328. In particular, FIG. 3A is a simplified schematic side view illustration of a portion of one embodiment of the balloon catheter 318 illustrated in a deflated state including an embodiment of the compensation assembly 328; and FIG. 3B is a simplified schematic side view illustration of the portion of the balloon catheter 318 illustrated in an inflated state including the compensation assembly 328 illustrated in FIG. 3A.

As shown, the balloon catheter 318 includes the guidewire lumen 332, the catheter shaft 334, and one or more balloons 336 (or other suitable expandable elements) that can be selectively moved between a deflated state (as shown in FIG. 3A) and an inflated state (as shown in FIG. 3B). It is appreciated that the guidewire 230 (illustrated in FIG. 2) is not shown substantially within the guidewire lumen 332 in FIGS. 3A and 3B for purposes of clarity.

Additionally, as provided herein, in various embodiments the compensation assembly 328 is configured to simultaneously and selectively adjust the position of the guidewire lumen 332 longitudinally as the one or more balloons 336 are moved between the deflated state and the inflated state. More specifically, in some such embodiments, the compensation assembly 328 is an automated, pressure-controlled and/or pressure-based compensation assembly that can be positioned at least partially within the handle assembly 220 (illustrated in FIG. 2) of the cryogenic balloon catheter system 210 (illustrated in FIG. 2). Alternatively, the compensation assembly 328 can be provided separately from the handle assembly 220.

As illustrated in FIGS. 3A and 3B, the catheter shaft 334 can be formed as an elongate body that is coupled at one end to a portion of the compensation assembly 328 and is coupled at the other end to the one or more balloons 336. Additionally, the guidewire lumen 332 can function as a core element that is positioned at least partially within the catheter shaft 334 and is movable relative to the catheter shaft 334. Further, a proximal end of the one or more balloons 336 can be coupled to a distal end of the catheter shaft 334, and a distal end of the one or more balloons 336 can be coupled near a distal end of the guidewire lumen 332.

The design of the compensation assembly 328 can be varied to suit the specific requirements of the cryogenic balloon catheter system 210 (illustrated in FIG. 2). As shown in the embodiment illustrated in FIGS. 3A and 3B, the compensation assembly 328 can include one or more of a fluid source 348, a fluid conduit 350, a housing 352, a slide 354 (or actuator), and a sealing element 356, e.g., a pneumatic sealing element. Additionally, or in the alternative, the compensation assembly 328 can include more components or fewer components than those specifically illustrated in FIGS. 3A and 3B.

In this embodiment, the compensation assembly 328 utilizes pressurized fluid 348A from the fluid source 348 to control the longitudinal movement of the guidewire lumen 332, e.g., relative to the catheter shaft 334 and/or relative to the housing 352 of the compensation assembly 328. More specifically, as described in detail herein, the fluid source 348 is configured to adjust a fluid pressure within the fluid conduit 350 and within the housing 352 (and thus within the one or more balloons 336, as such, the fluid pressure is sometimes also referred to as a “balloon pressure”), and to correspondingly control the longitudinal movement and position of the guidewire lumen 332 based on such fluid pressure. For example, a positive fluid pressure within the fluid conduit 350 and the housing 352 (and thus within the one or more balloons 336 such that the one or more balloons 336 are moved toward and/or are in the inflated state) will move the guidewire lumen 332 proximally, i.e. right-to-left as shown in the Figures, toward a retracted state. Conversely, a negative fluid pressure within the fluid conduit 350 and the housing 352 (and thus within the one or more balloons 336 such that the one or more balloons 336 are moved toward and/or are in the deflated state) will move the guidewire lumen 332 distally, i.e. left-to-right in the Figures, toward its fully extended state.

The fluid conduit 350 provides a fluid path to and from the fluid source 348. As shown in this embodiment, a portion of the fluid conduit 350 extends within the catheter shaft 334, and outside and adjacent to the guidewire lumen 332, between the housing 352 and the one or more balloons 336. Additionally, another portion of the fluid conduit 350 also extends within the housing 352 between the catheter shaft 334 and the slide 354. Stated in another manner, an interior of the housing 352 and the one or more balloons 336 are in fluid communication with the fluid source 348 via the fluid conduit 50. Alternatively, the fluid conduit 350 can be provided in another suitable manner.

The housing 352 is configured to remain substantially stationary during use of the balloon catheter 318. In the embodiment illustrated in FIGS. 3A and 3B, the housing 352 can be substantially annular-shaped to define a housing interior 360, and includes a first opening 352A and a second opening 352B. As shown, the first opening 352A is configured to receive a portion of the catheter shaft 334. Additionally, the first opening 352A is in fluid communication with the one or more balloons 336. The second opening 352B is in fluid communication with a reference environment, e.g., the (ambient) environment that surrounds the housing 352, the reference environment being at a reference pressure, e.g., an ambient pressure.

The slide 354 (or actuator) is positioned within the housing interior 360 of the housing 352 between the first opening 352A and the second opening 352B. It is appreciated that the positioning of the slide 354 within the housing interior 360 can be said to divide the housing interior 360 into a first interior region 360A and a second interior region 360B. As shown, the first interior region 360A is the portion of the housing interior 360 that extends between the slide 354 and the first opening 352A; and the second interior region 360B is the portion of the housing interior 360 that extends between the slide 354 and the second opening 352B.

Additionally, the slide 354 is movable longitudinally within the housing 352. As illustrated, the housing 352 can also include a first stop 358A (i.e. a distal stop) and a second stop 358B (i.e. a proximal stop) that are configured to limit the range of movement of the slide 354 within the housing 352 between a first (distal) position (when the slide 354 is at the first stop 358A) and a second (proximal) position (when the slide 354 is at the second stop 358B).

Further, the slide 354 also includes an opening 354A that is configured to receive a portion of the guidewire lumen 332. In particular, as shown, the guidewire lumen 332 is secured to the slide 354 and extends fully through and out of either end of the opening 354A that is formed into the slide 354. As such, longitudinal movement of the slide 354, as described herein, results in a corresponding longitudinal movement of the guidewire lumen 332.

The sealing element 356, e.g., a pneumatic sealing element, is positioned substantially between the slide 354 and the housing 352. Additionally, the sealing element 356 is configured to seal the connection, i.e. the movable connection, between the slide 354 and the housing 352 within the housing interior 360. With such design, fluid is inhibited from passing between the first interior region 360A and the second interior region 360B within the housing interior 360.

As provided herein, this embodiment of the compensation assembly 328 uses the fluid pressure created within the fluid conduit 350 (and as controlled by the fluid source 348 under the control of controller 361) to control movement of the guidewire lumen 332 relative to the housing 352. A positive fluid pressure (i.e. greater than the reference pressure) created within the fluid conduit 350 is used to inflate the one or more balloons 336. Additionally, the same positive fluid pressure within the fluid conduit 350 will act on the slide 354 and force the slide 354 to move proximally within the housing 352 until it reaches the proximal stop 358B which is preset as a desired final proximal position, i.e. the second position, for the slide 354. Accordingly, as the slide 354 moves proximally within the housing 352, the guidewire lumen 332 will also move proximally relative to the housing 352 until a desired preset maximum proximal position for the guidewire lumen 332 that coincides with the final proximal position, i.e. the second position, for the slide 354.

A negative fluid pressure (i.e. vacuum pressure that is lower than the reference pressure) created within the fluid conduit 350 is used to deflate the one or more balloons 336. As the one or more balloons 336 are deflated, it is desired to have the one or more balloons 336 be in their minimum profile which is a fully extended profile in the longitudinal direction. Additionally, the same negative fluid pressure within the fluid conduit 350 will act on the slide 354 and force the slide 354 to move distally within the housing 352 until it reaches the distal stop 358A which is preset as a desired final distal position, i.e. the first position, for the slide 354. Accordingly, as the slide 354 moves distally within the housing 352, the guidewire lumen 332 will also move distally relative to the housing 352 until a desired preset maximum distal position for the guidewire lumen 332 that coincides with the final distal position, i.e. the first position, for the slide 354.

It is appreciated that the housing 352, the slide 354 and the sealing element 356 can be formed from any suitable materials. For example, in some non-exclusive embodiments, the housing 352 and the slide 354 can be formed from one or more of a plastic material (e.g., polyether ether ketone (PEEK), polycarbonate, etc.) and/or a metal material (e.g., aluminum, stainless steel, etc.). Additionally, in certain non-exclusive embodiments, the sealing element 356 can be made of ethylene propylene diene monomer (EPDM) or other elastic materials. Alternatively, the housing 352, the slide 354 and the sealing element 356 can be formed from other suitable materials.

In summary, as provided herein, this embodiment of the present disclosure is configured to automatically synchronize the movement of the guidewire lumen 332 with the state, i.e. deflated or inflated, of the one or more balloons 336, without the need for operator involvement. Thus, the compensation assembly 328 is an automated, pressure-controlled compensation assembly that will move the guidewire lumen 332 proximally or distally based on the fluid pressure that is created within the fluid conduit

350. With such design, (i) the deflation of the one or more balloons 336 occurs automatically and simultaneously with the movement of the slide 354 (and the guidewire lumen 332) distally to the first position at the first stop 358A; and (ii) the inflation of the one or more balloons 336 occurs automatically and simultaneously with the movement of the slide 354 (and the guidewire lumen 332) proximally to the second position at the second stop 358B.

Additionally, in some embodiments, the cryogenic balloon catheter system 210 can further include a switch (not shown) for use by the operator to determine when to inflate or deflate the one or more balloons 336, or to selectively move the one or more balloons 336 between the deflated state and the inflated state. In such embodiments, the fluid source 348, under control of the controller 361, will provide a positive pressure for inflation or a negative pressure for deflation based on the signal from the switch. The automatic compensation system will act accordingly to move the guidewire lumen proximally or distally based on the pressure.

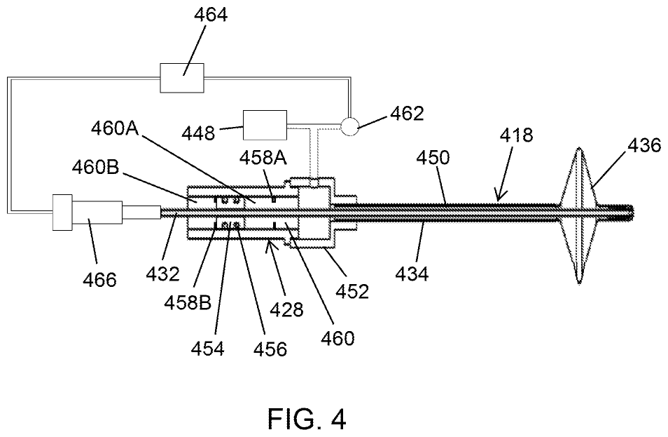

FIG. 4 is a simplified schematic side view illustration of a portion of another embodiment of the balloon catheter 418 illustrated in the inflated state including another embodiment of the compensation assembly 428. The balloon catheter 418 is substantially similar to what has been previously described herein. More particularly, the balloon catheter 418 again includes the guidewire lumen 432, the catheter shaft 434, the one or more balloons 436 (or other suitable expandable elements) that can be selectively moved between a deflated state and an inflated state, and the compensation assembly 428.

Additionally, the compensation assembly 428 is somewhat similar to what was illustrated and described above in the embodiment shown in FIGS. 3A and 3B. For example, the compensation assembly 428 again includes a fluid source 448, a fluid conduit 450, a housing 452, a slide 454 and a sealing element 456 that are substantially similar to what was illustrated and described in the previous embodiment. However, in this embodiment, the compensation assembly 428 further includes a pressure sensor 462, a controller 464 and an actuator 466. As provided herein, the compensation assembly 428 illustrated in this embodiment again is utilized to automatically and simultaneously adjust the longitudinal position of the guidewire lumen 432 relative to the housing 452 as the one or more balloons 436 are moved between the deflated state and the inflated state.

As with the previous embodiment, the fluid source 448 is in fluid communication with the one or more balloons 436. In the embodiment shown in FIG. 4, the pressure sensor 462 is configured to sense or measure a pressure within the one or more balloons 436, and to generate a pressure signal that is based on the sensed pressure within the one or more balloons 436. Because the fluid conduit 450 and the first interior region 460A of the housing interior 460 are also in fluid communication with the fluid source 448, the pressure within the fluid conduit 450 and within the first interior region 460A of the housing interior 460 is substantially equal to the pressure within the one or more balloons 436. Additionally, as with the previous embodiment, the pressure within the one or more balloons 436, and thus within the first interior region 460A of the housing interior 460, will function to move the slide 454 longitudinally, i.e. either proximally or distally, within the housing 452 depending on whether the pressure within the first interior region 460A is higher or lower than the reference pressure, e.g., the ambient environmental pressure, within the second interior region 460B of the housing interior 460.

However, in this embodiment, the movement of the slide 454 within the housing 460 will be further aided by use of the actuator 466, which, as shown, is coupled to the guidewire lumen 432. In particular, as noted, the pressure sensor 462 is configured to sense the pressure within the one or more balloons 436, and to generate a pressure signal that is based on the sensed pressure. The pressure signal is then sent to the controller 464, which is electrically coupled to both the pressure sensor 462 and the actuator 466. The controller 464 uses the pressure signal to drive the actuator 466 in either direction to aid the movement of the guidewire lumen 432 and the slide 454. For example, if the sensed pressure is negative relative to the reference pressure, and the one or more balloons 436 are deflated, the controller 464 will use the sensed pressure to drive to actuator 466 distally. This movement of the actuator 466 will further push the guidewire lumen 432, and thus the slide 454 to which the guidewire lumen 432 is secured, distally toward the first position, i.e. such that the slide 454 is moved toward the first stop 458A. Conversely, if the sensed pressure is positive relative to the reference pressure, and the one or more balloons 436 are inflated, the controller 464 will use the sensed pressure to drive the actuator 466 proximally. This movement of the actuator 466 will further move the guidewire lumen 432, and thus the slide 454 to which the guidewire lumen 432 is secured, proximally toward the second position, i.e. such that the slide 454 is moved toward the second stop 458B. It is appreciated that the predetermined stop positions are utilized to inhibit undesired stress on the guidewire lumen 432.

It is appreciated that the actuator 466 can have any suitable design for purposes of moving the guidewire lumen 432 between the first position and the second position. For example, in certain non-exclusive embodiments, the actuator 466 can be an electrical linear actuator, a gas cylinder, a step motor with linear movement mechanism, or a solenoid actuator. Alternatively, the actuator 466 can have another suitable design. Additionally, methods to operate the actuator 466 can be a predetermined sequence, a confirmation through a switch, a touch button, a remote keypad or a touch screen, etc.

Thus, this embodiment of the compensation assembly 428 again provides an automated, pressure-controlled and/or pressure-based compensation assembly that is configured to automatically synchronize the movement of the guidewire lumen 432 with the state, i.e. deflated or inflated, of the one or more balloons 436, without the need for operator involvement.

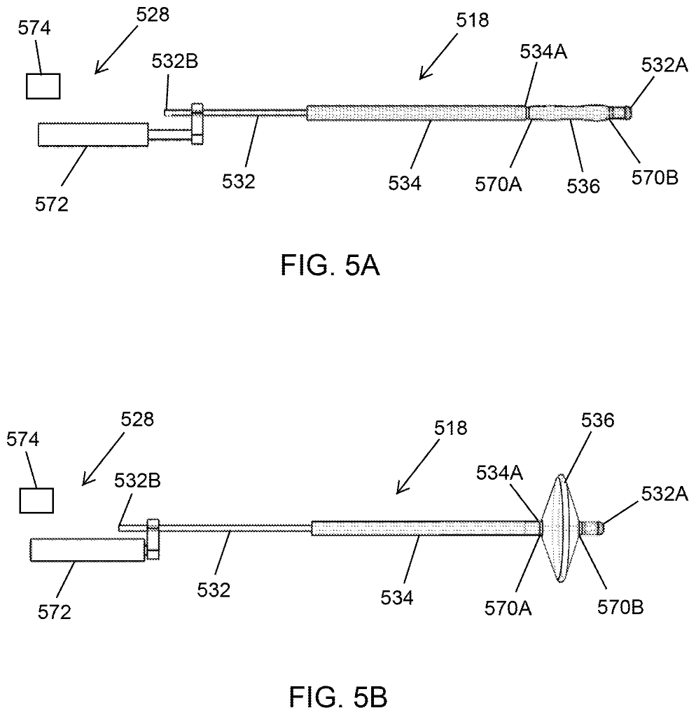

FIGS. 5A and 5B illustrate the design and operation of still another non-exclusive embodiment of the compensation assembly 528. In particular, FIG. 5A is a simplified schematic side view illustration of a portion of still another embodiment of the balloon catheter 518 illustrated in the deflated state including still another embodiment of the compensation assembly 528; and FIG. 5B is a simplified schematic side view illustration of the portion of the balloon catheter 518 illustrated in the inflated state including the compensation assembly 528 illustrated in FIG. 5A.

As shown, the balloon catheter 518 is somewhat similar to what has been previously described herein. More particularly, the balloon catheter 518 again includes the guidewire lumen 532, the catheter shaft 534, the one or more balloons 536 (or other suitable expandable elements) that can be selectively moved between a deflated state and an inflated state, and the compensation assembly 528. Additionally, as above, a proximal end 570A of the one or more balloons 536 is coupled to a distal end 534A of the catheter shaft 534, and a distal end 570B of the one or more balloons 536 is coupled near a distal end 532A of the guidewire lumen 532. Further, the guidewire lumen 532 is again positioned or disposed at least partially within the catheter shaft 534, and is movable within the catheter shaft 534.

However, in this embodiment, the compensation assembly 528 is somewhat different than what was illustrated and described in the previous embodiments. In particular, as illustrated, the compensation assembly 528 includes an actuator 572 and a controller 574. Alternatively, the compensation assembly 528 can include more or fewer components than what is specifically shown in FIGS. 5A and 5B.

In this embodiment, the actuator 572 is fixedly coupled to the guidewire lumen 532, i.e. near a proximal end 532B of the guidewire lumen 532. Additionally, the actuator 572 is configured to selectively move the guidewire lumen 532 longitudinally in a first direction or an opposed second direction under the control of the controller 574. More specifically, in certain embodiments, the actuator 572 can be controlled by the controller 574 to compensate for necessary changes in length and/or positioning of the guidewire lumen 532 just prior to, just subsequent to, and/or contemporaneous with any selective inflation and deflation of the one or more balloons 536. As such, the selective movement of the guidewire lumen 532 by the actuator 572 under control of the controller 574 can be said to substantially coincide with the movement of the one or more balloons 536 between the inflated state and the deflated state.

FIG. 5A illustrates the actuator 572 in an extended state such that the one or more balloons 536 are in their minimum profile which is a fully extended profile in the longitudinal direction. It is also appreciated that the guidewire lumen 532 has been moved to its most distal position when the actuator 572 is in the extended state. Stated in another manner, movement of the actuator 572 toward its extended state results in corresponding movement of the guidewire lumen 532 in the distal direction. It is further appreciated that it is preferred that the balloons 536 be in their minimum and fully extended profile prior to insertion of the balloon catheter 518 into the patient, prior to removal of the balloon catheter 518 from the patient, prior to repositioning of the balloon catheter 518 within the patient, and any other time when it is desired that the one or more balloons 536 be in the deflated state.

FIG. 5B illustrates the actuator 572 in a retracted state and the one or more balloons 536 in the inflated state. It is also appreciated that the guidewire lumen 532 has been moved to its most proximal position when the actuator 572 is in the retracted state. Stated in another manner, movement of the actuator 572 toward its retracted state results in corresponding movement of the guidewire lumen 532 in the proximal direction. In this position, the one or more balloons 536 are (or can be) inflated, and the desired cryoablation procedure can be performed by the operator.

In certain applications, when it is desired to move the one or more balloons 536 from the inflated state to the deflated state, the steps can be performed essentially as follows, although it is understood that the order of the steps can be changed as desired. In one step, the controller 574 sends a signal to the actuator 572 to activate the actuator 572. In another step, under control of the controller 574, the actuator 572 moves the guidewire lumen 532 distally to a predetermined distal position (or first position), resulting in a fully extended state of the one or more balloons 536. In still another step, the controller 574 then initiates a deflation of the one or more balloons 536 with a termination of fluid flow and an evacuation of the one or more balloons 536. Conversely, when it is desired to move the one or more balloons 536 from the deflated state to the inflated state, the following steps can be performed. In one step, the controller 574 sends a signal to the actuator 572 to activate the actuator 572. In another step, under control of the controller 574, the actuator 572 moves the guidewire lumen 532 proximally to a predetermined proximal position (or second position). In still another step, the controller 574 initiates an inflation of the one or more balloons 536 with an initiation of fluid flow into the one or more balloons 536.

It is appreciated that either of the predetermined positions of the guidewire lumen 532 during the longitudinal movement of the guidewire lumen 532 by the actuator 572 can be referred to as a “first position” or a “second position”.

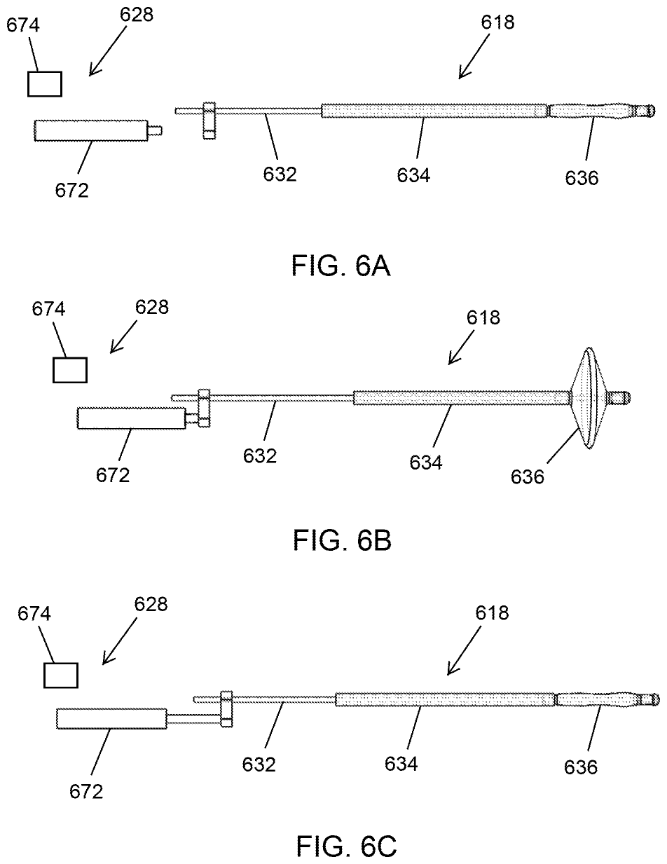

FIGS. 6A-6C illustrate the design and operation of yet another non-exclusive embodiment of the compensation assembly 628. In particular, FIG. 6A is a simplified schematic side view illustration of a portion of yet another embodiment of the balloon catheter 618 illustrated in the deflated state including yet another embodiment of the compensation assembly 628; FIG. 6B is a simplified schematic side view illustration of the portion of the balloon catheter 618 illustrated in the inflated state including the compensation assembly 628 illustrated in FIG. 6A; and FIG. 6C is a simplified schematic side view illustration of the portion of the balloon catheter 618 again illustrated in the deflated state including the compensation assembly illustrated 628 in FIG. 6A.

As shown, the balloon catheter 618 is substantially similar to what was illustrated and described herein above in relation to FIGS. 5A and 5B. More particularly, the balloon catheter 618 again includes the guidewire lumen 632, the catheter shaft 634, the one or more balloons 636 (or other suitable expandable elements) that can be selectively moved between a deflated state and an inflated state, and the compensation assembly 628. Additionally, as shown, the compensation assembly 628 again includes the actuator 672 and the controller 674 that are similar in design and general functionality to the previous embodiment. However, in this embodiment, the compensation assembly 628 operates in a somewhat different manner in that the actuator 672 is only selectively coupled, i.e. removably coupled, to the guidewire lumen 632. Thus, the actuator 672, under control of the controller 674, will only control movement of the guidewire lumen 632 at certain times during use of the balloon catheter 618.

For example, in one non-exclusive application, the compensation assembly 628 can be configured to only control movement of the guidewire lumen 632 during a deflation of the one or more balloons 636, i.e. when the one or more balloons 636 are moved from the inflated state to the deflated state. As shown in FIG. 6A, with the one or more balloons 636 already being in the deflated state, the actuator 672 is shown in a non-contact position, i.e. the actuator 672 is not coupled to the guidewire lumen 632. At such time, the one or more balloons 636 can be selectively inflated, i.e. moved from the deflated state to the inflated state, as desired, under control of the controller 674. Subsequently, the actuator 672 can then be selectively coupled to the guidewire lumen 632 at the end of the inflation process. The actuator 732 being coupled to the guidewire lumen 632 with the one or more balloons 636 being in the inflated state is shown in FIG. 6B.

When it is then desired to return the one or more balloons 636 to the deflated state, the actuator 672, under control of the controller 674, can move the guidewire lumen 632 distally so that the one or more balloons 636 are at their fully extended state. Additionally, the one or more balloons 636 can be selectively deflated, i.e. moved from the inflated state to the deflated state, as desired, under control of the controller 674. It is appreciated that in alternative embodiments, the movement of the guidewire lumen 632 can be performed prior to the movement of the one or more balloons 636 to the deflated state, subsequent to the one or more balloons 636 being moved to the deflated state, or substantially simultaneously with the one or more balloons 636 being moved to the deflated state. Once the deflation process has been completed, the actuator 672 can again be uncoupled from the guidewire lumen 632. FIG. 6C illustrates that the one or more balloons 636 have been moved back to the deflated state, and the actuator 672 is ready to be uncoupled from the guidewire lumen 632.

In alternative applications, the compensation assembly 628 can additionally or alternatively be configured to control movement of the guidewire lumen 632 during an inflation of the one or more balloons 636, i.e. when the one or more balloons 636 are moved from the deflated state to the inflated state.

It is understood that although a number of different embodiments of the compensation assembly 228 of the cryogenic balloon catheter system 210 have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A compensation assembly for a balloon catheter system, the balloon catheter system including a balloon catheter having a guidewire lumen and a balloon that is secured to the guidewire lumen, the balloon being movable between a deflated state and an inflated state, the compensation assembly comprising:
a housing including a housing interior;
a slide that is secured to the guidewire lumen, the slide being positioned within the housing interior and being movable relative to the housing; and
a fluid source that is in fluid communication with the balloon via a fluid conduit, the fluid source being controlled to selectively adjust a balloon pressure within the balloon so that the balloon moves between the deflated state and the inflated state;
wherein the balloon pressure moves the slide relative to the housing in (i) a first direction when the balloon is moved from the inflated state to the deflated state, and (ii) a second direction when the balloon is moved from the deflated state to the inflated state.

2. The compensation assembly of claim 1 wherein the first direction is substantially opposite to the second direction.

3. The compensation assembly of claim 1 wherein the housing interior includes a first interior region that is in fluid communication with the fluid source via the fluid conduit, and a second interior region that is at a reference pressure.

4. The compensation assembly of claim 3 wherein the slide is positioned between the first interior region and the second interior region.

5. The compensation assembly of claim 3 wherein the fluid source selectively provides a negative balloon pressure that is lower than the reference pressure to move the balloon toward the deflated state and simultaneously move the slide relative to the housing in the first direction.

6. The compensation assembly of claim 5 wherein the fluid source selectively provides a positive balloon pressure that is greater than the reference pressure to move the balloon toward the inflated state and simultaneously move the slide relative to the housing in the second direction.

7. The compensation assembly of claim 1 further comprising a sealing element that seals a connection between the slide and the housing within the housing interior.

8. The compensation assembly of claim 8 wherein the sealing element is a pneumatic sealing element.

9. A compensation assembly for a balloon catheter system, the balloon catheter system including a balloon catheter having a guidewire lumen and a balloon that is secured to the guidewire lumen, the balloon being movable between a deflated state and an inflated state, the compensation assembly comprising:
a housing including a housing interior;
a slide that is secured to the guidewire lumen, the slide being positioned within the housing interior and being movable relative to the housing;
a fluid source that is in fluid communication with the balloon via a fluid conduit, the fluid source being controlled to selectively adjust a balloon pressure within the balloon so that the balloon moves between the deflated state and the inflated state; and
a pressure sensor that is configured to sense the balloon pressure, the pressure sensor generating a pressure signal that is based on the sensed pressure;
wherein the balloon pressure moves the slide relative to the housing in (i) a first direction when the balloon is moved from the inflated state to the deflated state, and (ii) a second direction when the balloon is moved from the deflated state to the inflated state.

10. The compensation assembly of claim 9 further comprising an actuator that is coupled to the guidewire lumen.

11. The compensation assembly of claim 10 further comprising a controller that is electrically coupled to the pressure sensor and the actuator, the controller being configured to receive the pressure signal and to move the guidewire lumen based at least partially upon the pressure signal.

12. The compensation assembly of claim 9 wherein the housing interior includes a first interior region that is in fluid communication with the fluid source via the fluid conduit, and a second interior region that is at a reference pressure.

13. The compensation assembly of claim 12 wherein the slide is positioned between the first interior region and the second interior region.

14. The compensation assembly of claim 12 wherein the fluid source selectively provides a negative balloon pressure that is lower than the reference pressure to move the balloon toward the deflated state and simultaneously move the slide relative to the housing in the first direction.

15. The compensation assembly of claim 9 wherein the housing includes a first stop that limits movement of the slide relative to the housing in the first direction, and a second stop that limits movement of the slide relative to the housing in the second direction.

16. The compensation assembly of claim 9 wherein at least a portion of the fluid conduit is positioned adjacent to the guidewire lumen.

17. A compensation assembly for a balloon catheter system, the balloon catheter system including a balloon catheter having a guidewire lumen and a balloon that is secured to the guidewire lumen, the balloon being movable between a deflated state and an inflated state, the compensation assembly comprising:
an actuator that is coupled to the guidewire lumen and moves the guidewire lumen between a first position and a second position; and
a controller that controls the actuator such that the actuator moves the guidewire lumen between the first position and the second position so as to substantially coincide with the movement of the balloon between the deflated state and the inflated state.

18. The compensation assembly of claim 17 wherein the controller controls the actuator to move the guidewire lumen between the first position and the second position just prior to the movement of the balloon between the deflated state and the inflated state.

19. The compensation assembly of claim 17 wherein the controller controls the actuator to move the guidewire lumen between the first position and the second position just subsequent to the movement of the balloon between the deflated state and the inflated state.

20. The compensation assembly of claim 17 wherein the controller controls the actuator to move the guidewire lumen between the first position and the second position substantially contemporaneous with the movement of the balloon between the deflated state and the inflated state.
